(19) United States
(12) Patent Application Publication
(10) Pub. No.: US 2021/0330936 A1
(43) Pub. Date: Oct. 28, 2021

(54) FLOW INDWELLING URINARY CATHETER

(71) Applicant: Mohamed Hussein Higgy, Redwood City, CA (US)

(72) Inventor: Mohamed Hussein Higgy, Redwood City, CA (US)

(21) Appl. No.: 16/859,747

(22) Filed: Apr. 27, 2020

(51) Int. Cl. A61M 25/00; A61M 25/10

(52) U.S. Cl. CPC A61M 25/0017; A61M 25/10; A61M 2210/1085; A61M 25/0068; A61M 2210/1089; A61M 25/0026

(57) ABSTRACT

Improved flow indwelling urinary catheters are disclosed in which inflow openings at a distal end of a catheter are distally biased and in proximity to a distal end of a catheter balloon resulting in greater urine outflow volume and velocity, greater urine emptying volume from the bladder and patent urine outflow as the catheter is withdrawn from the bladder.

### BACKGROUND OF THE INVENTION

The present invention pertains generally to urethral catheters and more particularly to improved flow indwelling urinary catheters. Urinary catheters have been available and used for decades to facilitate draining urine from a bladder in situations where a person is incontinent, has urethral obstructions, such as strictures, or to assist in urine drainage where the bladder muscle is not functioning normally. Indwelling urinary catheters are typically delivered through the urethra and into the bladder. A distal end of the catheter is positioned above the bladder neck and a small balloon is inflated to retain the catheter in the bladder. Conventional urinary catheters, such as a Foley catheter, have become ubiquitous in the art and, typically, have a distal end with flow ports passing through side walls of the distal end of the catheter. These flow ports are typically positioned proximal to a distal tip of the catheter, leaving a relatively large distal portion of the catheter and typically have openings that are oriented perpendicular to the longitudinal axis of a flow lumen in the catheter with which the openings communicate.

A major shortcoming of conventional indwelling urinary catheters are that the inflow openings are either positioned a relatively great distance distally from the balloon and open perpendicular to the longitudinal axis of the drainage lumen in the catheter. This configuration leads to inefficient bladder drainage and occlusion of the inflow openings by the urethral walls as the catheter is being withdrawn from the bladder.

A recent attempt to address some of these disadvantages of the Foley catheter has been undertaken by The Flume Catheter Company (Suffolk, Great Britain) as exemplified in U.S. Pat. No. 10,195,394. The urine inflow openings of the FLUME catheter are positioned much closer to the proximal end of a channeled balloon than is found in the Foley catheter and some distance from the distal tip of the catheter. This position of the urine inflow openings, however, has been found to become easy occluded as the balloon is deflated for withdrawal or either or both of the balloon channels is not clear to allow urine flow to the urine inflow opening. Both of these situations lead to disadvantageous cessation of urine flow and inadequate bladder emptying.

### SUMMARY OF THE INVENTION

It is an objective of the present invention to provide an indwelling urinary catheter in which inflow openings communicating with a catheter flow lumen are positioned a relatively minor distance distally from a catheter balloon.

It is a further object of the present invention to provide an indwelling urinary catheter in which the inflow openings are oriented on a distal facing bias and non-perpendicular relative to the longitudinal axis of the flow lumen.

It is a still further objective of the present invention to provide an indwelling urinary catheter having a generally conical distal tip, wherein the inflow openings are in the generally conical distal tip.

It is another objective of the present invention to provide an indwelling urinary catheter having a primary lumen for urine drainage and a secondary lumen for balloon inflation.

It is yet a further objective of the present invention to provide an indwelling urinary catheter having a tertiary lumen for delivery or withdrawal of fluids, such as drugs, that extends from a proximal end of the indwelling urinary catheter and open at a distal end of the indwelling urinary catheter.

It is yet a still further objective of the present invention to provide an indwelling urinary catheter that maintains patency of fluid flow into the flow lumen as the balloon is deflated and the catheter is withdrawn through the urethra.

It is yet another objective of the present invention to provide an indwelling urinary catheter that is configured to allow an increased volume of urine drainage from the bladder when it is placed in the bladder.

It is a further objective of the present invention to provide an indwelling urinary catheter that is configured to allow for an increased rate of urine drainage from the bladder when it is placed in the bladder.

It is still another objective of the present invention to provide an indwelling urinary catheter having a balloon with a shape and inflation capacity that is configured to decrease the bladder insertion distance of the catheter to achieve urine drainage from the bladder.

It is yet still another objective of the present invention to provide an indwelling urinary catheter wherein the balloon shape, inflation capacity, and positioning of the urine inflow openings of the urinary catheter are configured to increase the urine drainage volume and drainage velocity from the bladder, while increasing patient comfort while the urinary catheter is in place in the bladder.

It is still another objective of the present invention to provide a method of draining urine from a urinary bladder in which the urinary bladder is at least substantially emptied of urine and without appreciable urine load remaining within the bladder.

It is yet another objective of the present invention to provide a method of draining urine from a urinary bladder that includes the steps of placing the inventive indwelling urinary catheter within a urinary bladder through the urethra, inflating the balloon thereby securing the indwelling urinary catheter in the bladder and positioning the urine inflow openings superior to and adjacent a distal of the balloon, draining urine through the urine inflow openings and into a urine drainage lumen within the indwelling urinary catheter, and upon removal of the indwelling urinary catheter, maintaining urine flow into the urine inflow openings during removal of the indwelling urinary catheter to at least substantially empty the urinary bladder.

### BRIEF DESCRIPTION OF THE DRAWINGS

FIG. 1 is a perspective of a first embodiment of the indwelling urinary catheter in accordance with the present invention.

FIG. 2 is a side elevational view of the first embodiment of the indwelling urinary catheter in accordance with the present invention.

FIG. 2A is a cross-sectional view taken along line 2A-2A of FIG. 2.

FIG. 3 is a first side elevational view of a distal end of the indwelling urinary catheter in accordance with the present invention.

FIG. 4 is a second side elevational view of the distal end of the indwelling urinary catheter in accordance with the present invention.

FIG. 5A is a side elevational view of the distal end of the indwelling urinary catheter of the present invention illustrating a balloon in its inflated state.

FIG. 5B is a cross-sectional view taken along line 5B-5B of FIG. 5A.

FIG. 5C is a side elevational view of a distal end of a conventional indwelling urinary catheter in its inflated state.

FIG. 5D is a cross-sectional view taken along line 5D-5D of FIG. 5C.

FIG. 6 is a perspective view of a second embodiment of the indwelling urinary catheter in accordance with the present invention.

FIG. 7 is a fragmentary view taken along circle 7 of FIG. 6.

FIG. 8 is a diagrammatic illustration of a conventional Foley catheter placed in a bladder illustrating urine flow into the catheter.

FIG. 9 is a diagrammatic illustration of the present invention placed in a bladder illustrating urine flow into the inventive indwelling urinary catheter.

FIG. 10 is a diagrammatic illustration of a conventional Foley catheter placed in a bladder illustrating urine drainage level in the bladder.

FIG. 11 is a diagrammatic illustration of the present invention placed in a bladder illustrating urine drainage level with the inventive indwelling urinary catheter.

FIG. 12 is a diagrammatic illustration of a bladder showing occlusion of urine flow during removal of a conventional indwelling urinary catheter.

FIG. 13 is a diagrammatic illustration showing continuous urine flow during removal of the indwelling urinary catheter of the present invention.

FIG. 14 is a diagrammatic illustration showing urinary bladders and comparative urine drainage levels between a prior art Foley catheter and the indwelling urinary catheter of the present invention.

FIG. 15 is a diagrammatic side elevational view of the indwelling urinary catheter of the present invention illustrating dimensional variables.

### DETAILED DESCRIPTION OF THE PREFERRED EMBODIMENTS

The terminology used herein is for the purpose of describing example embodiments only and is not intended to be limiting. For purposes of clarity, the following terms used in this patent application will have the following meanings:

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

When an element or layer is referred to as being “on,” “engaged,” “connected,” or “coupled” to or with another element, it may be directly on, engaged, connected or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to totally. For example, substantially vertical may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

Recitation of ranges of values herein are merely intended to serve as a shorthand method of referring individually to each separate value falling within the recited range, unless otherwise indicated herein, and each separate value is incorporated into the specification as if it were individually recited herein.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, biomedical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

This detailed description of exemplary embodiments makes reference to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

The present invention includes both an indwelling urinary catheter 10 and a method of at least substantially emptying a urine load from a urinary bladder without an appreciable urine load remaining within the bladder. The method In accordance with the method of the present invention, The method of draining urine from a urinary bladder includes the steps of placing the inventive indwelling urinary catheter within a urinary bladder through the urethra, inflating the balloon thereby securing the indwelling urinary catheter in the bladder and positioning the urine inflow openings superior to and adjacent a distal of the balloon, draining urine through the urine inflow openings and into a urine drainage lumen within the indwelling urinary catheter, and upon removal of the indwelling urinary catheter, maintaining urine flow into the urine inflow openings during removal of the indwelling urinary catheter to at least substantially empty the urinary bladder.

Turning now to the accompanying Figures in which exemplary embodiments of the present invention are illustrated and common reference numerals denote common features among the exemplary embodiments. Two main embodiments of the urinary catheter of present invention are illustrated in the accompanying Figures. A first embodiment is a two-way catheter 10, having a urine flow lumen and a balloon inflation lumen and a second embodiment is a three-way catheter 30, having a three lumens, including a urine flow lumen, a balloon inflation lumen, and a tertiary lumen.

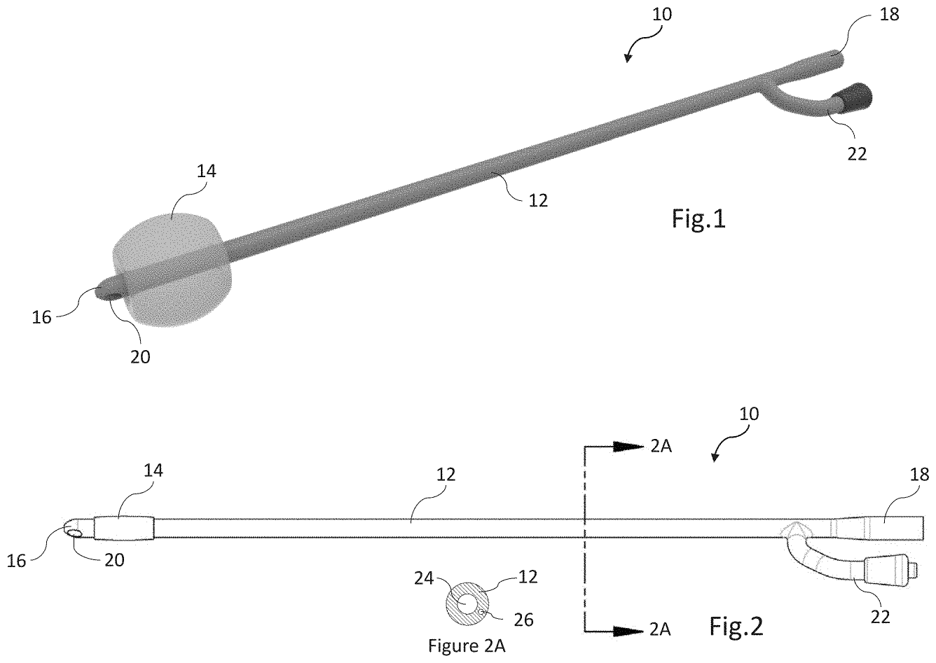

As illustrated in FIGS. 1-4 catheter 10 consist generally of two-way catheter having catheter body 12 which is an elongate tubular member having a drainage lumen 24 and the balloon inflation lumen 26 (See, FIG. 2A), extending through the catheter body 12. A balloon 14, is affixed to the catheter body 12 at a distal portion thereof. The balloon 14 is typically concentrically affixed to the catheter body 12 and inflates in a substantially toroidal spherical shape as a fluid, typically saline, is injected into the balloon 14. The balloon inflation lumen 26 is in fluid flow communication with balloon 14 and with an inflation connector 22 positioned at a proximal end of the catheter body 12. Typically, urinary catheter balloons 14 have a maximum balloon inflation capacity between about 1.5 ml to about 35 ml, where smaller balloons are employed in pediatric applications and larger balloons are employed in adults.

A drainage port 18 is in fluid flow communication with the drainage lumen 24, shown in FIG. 2A, and is configured to accommodate coupling to a urine drainage line at a proximal end of the catheter body 12. At least one, and preferably at least two, inflow openings 20 are provided at a distal tip 16 of the catheter body 12. The inflow openings 20 communicate with the drainage lumen 24 at a distal portion of the catheter body 12. The inflow openings 20 have a distal facing bias relative to the longitudinal axis 13 of the catheter body 12. The distal tip 16 of the catheter body 12 preferably has a substantially conical or frustoconical shape in the inflow openings 20 are positioned in diametric opposition relative to each other in the distal tip 16.

While the inflow openings 20 may have a wide variety of transverse opening shapes in the plane of the distal tip 20, to maximize the open surface area of each of the inflow openings 20, the inflow openings 20 preferably have a transverse opening shape that conforms to the conical shape of the distal tip 16. The transverse opening shape of the inflow openings 20 may be substantially circular, elliptical, ovoidal, triangular, or the like that is configured to maximize the open area and urine inflow into the drainage lumen 24. In this manner, the open surface area of the inflow openings 20 at the surface of the distal tip 16 is configured to allow the greatest opening configuration to permit urine inflow into the inflow openings 20.

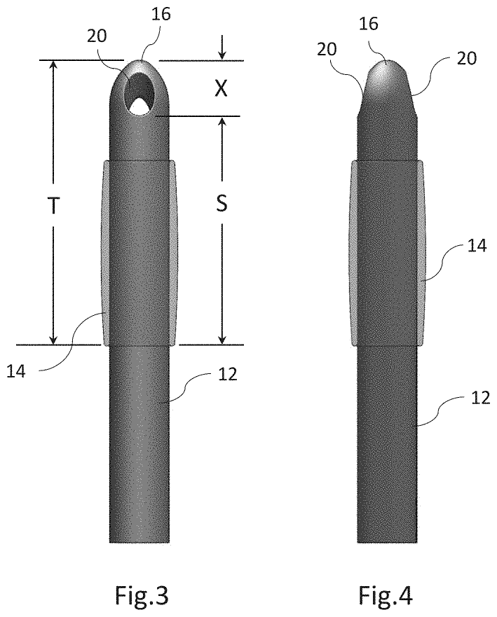

With particular reference to FIG. 3, there is shown the distal portion of indwelling urinary catheter 10 in which the balloon 14 is deflated. The positioning of the inflow openings 20 in the distal tip 16 relative to the balloon 14 is significant in the inventive urinary catheter 10. Distance S is the measurement taken from a proximal end of uninflated balloon 14 to a proximal end of inflow opening 20. Distance T is the measurement taken from the proximal end of uninflated balloon 14 to the distal end of distal tip 16. It will be understood, therefore, that the distance X between the proximal end of inflow opening 20 and the distal end of distal tip 16 is expressed by the equation:

X=T−S

When compared to a standard urinary catheter such as a Foley catheter, the values of each of T, S, and X, are all less than that found in a standard urinary catheter. As an example, in a standard Foley catheter, distance T is typically about 53 mm for a two-way catheter whereas it is about 55 mm for a three-way Foley catheter, whereas in the present invention, as illustrated distance T is obtained by adding K+L+7 and is between about 32 to about 36 mm depending on the balloon volume. Further, in a standard Foley catheter, distance S is typically about 37 mm for the two-way catheter and 39 mm for the three-way catheter, whereas in the present invention distance S is obtained by adding K+L and is between about 25 to about 29 mm, again depending on balloon volume. Following the above equation, the distance X, between the proximal end of the flow opening and the distal end of the distal tip, is about 16 mm in a standard Foley catheter and about 7 mm in the urinary catheter 10 of the present invention. The foregoing dimensions are with reference to an uninflated balloon. It will be understood that when the balloon is inflated in the bladder, the proximal end of the balloon will extend between about 0 to 4 mm further depending upon the inflated balloon volume. Inflated balloon volumes may range from 15 ml to 30 ml, as shown in Table 1, below.

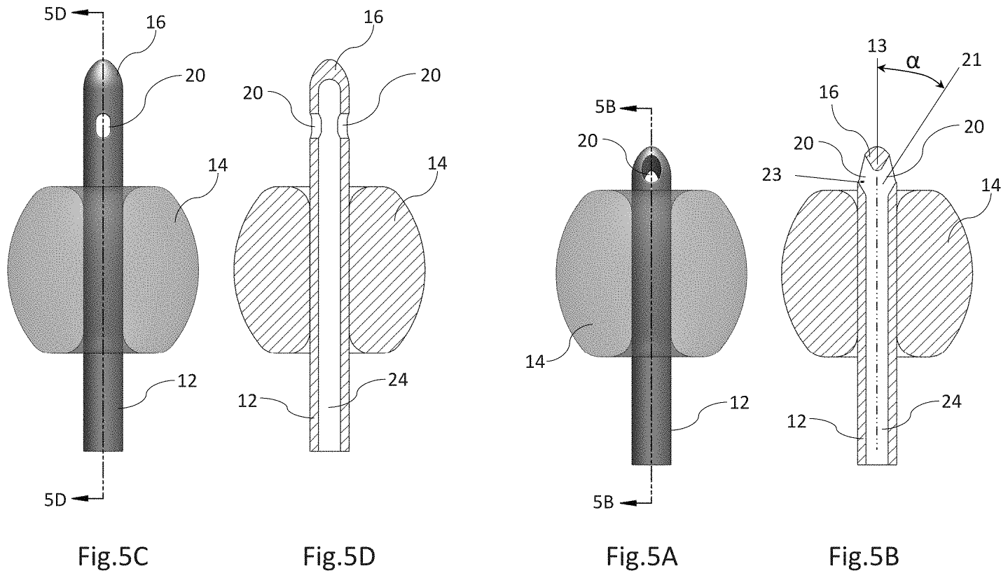

As noted above, each of the inflow openings 20 has a distal facing bias. As shown in FIG. 5B, the distal bias 21 of the inflow openings 20 forming angle α relative to the longitudinal axis 13 of the catheter body 12. Angle α is between about 15 to about 75° relative to the longitudinal axis 13 of the catheter body 12, preferably angle α is between about 20 to about 45° and most preferably it is between about 25 to about 35°. It will be understood that angle α of the inflow openings 20 affects the urine flow profile into and through the inflow openings 20 and by having the inflow openings 20 with a distal bias of angle α, the inflow openings 20 facilitate optimal flow of urine from the bladder and into the drainage lumen 24 of catheter body 12.

It will be understood by those skilled in the art, therefore, that with both the two-way and three-way variants the indwelling urinary catheter 10, 30, respectively, of the present invention, the distal tip 16 and the inflow openings 20 are in far greater proximity to a distal end of balloon 14 than is found in conventional urinary catheters. This relatively closer approximation between the balloon 14 and the distal tip 16, positions the inflow openings 20 closer to the bladder neck relative to standard urinary catheters when the urinary catheter is properly positioned within the bladder. FIGS. 5A and 5B more clearly illustrate the proximity between the distal end of balloon 14 and the inflow openings 20 in the distal tip 16 when the balloon 14 is in its expanded state. It will be understood that the proximal end of at least one of the inflow openings 20 is positioned at substantially at the same level as or below the distal end of balloon 14. FIGS. 5C and 5D illustrate a conventional urinary catheter and the greater distance S, defined above, between the distal end of the inflated balloon 14 and the proximal end of inflow opening 20 than that found in the indwelling urinary catheter 10 of the present invention. As is shown in FIGS. 5C and 5D, in a conventional urinary catheter, the proximal end of the inflow openings 20 is distally spaced and not substantially at the same level as the distal end of balloon 14.

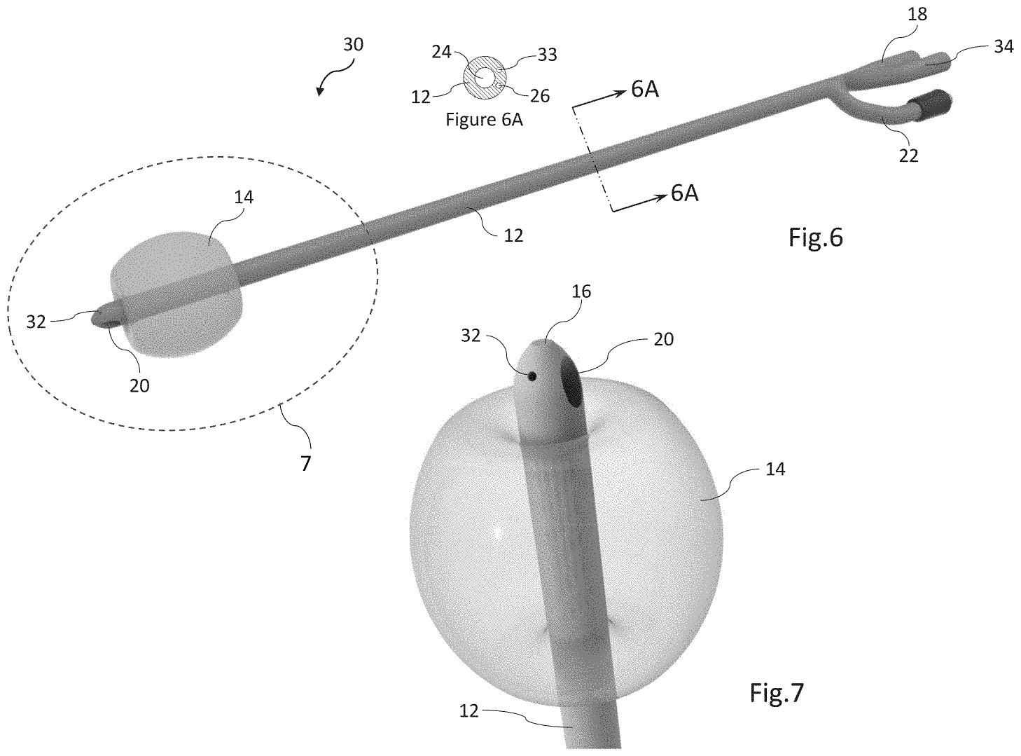

A second embodiment of the indwelling urinary catheter 30 is illustrated in FIG. 6. Indwelling urinary catheter 30, which is a three-way catheter, is substantially identical to indwelling urinary catheter 10, with the exception that a tertiary lumen (not shown) is provided, which may be employed to deliver or withdraw fluids, such as drugs or urine samples, respectively. The tertiary lumen passes along the longitudinal axis of the catheter body 12 and communicates with a fluid port 32 at the distal end of the tertiary lumen and with a fluid connector 34 at a proximal end of the catheter body 12. One skilled in the art will understand that the tertiary lumen (not shown) is similar to the inflation lumen 26 in FIG. 2A and extends from the fluid port 32 to the fluid connector 34. The fluid port 32 passes through the distal tip 16 and is positioned between the inflow openings 20. Fluid port 32, tertiary lumen (not shown) and fluid connector 34 are configured to allow a syringe or other vessel to be coupled to the fluid connector 34 and deliver a pharmaceutically active agent into and through the tertiary lumen 36 and out the fluid port 32 and into the bladder. Similarly, fluids, such as urine samples, may be withdrawn through the fluid port. The term “connector” is intended to encompass different mechanical or other types of engagement configurations that allow for a syringe or other vessel to be fluidly coupled to the tertiary lumen (not shown) to deliver a fluid from the syringe or other vessel to the tertiary lumen. Examples of suitable connectors include a luer fitting, a friction fit connector, a snap fit, a one-way valve, or the like. FIG. 7 is an enlarged view along circle 7 of FIG. 6 and more clearly illustrates the positioning of the fluid port 32 in the distal tip 16 and relative to the inflow opening 20.

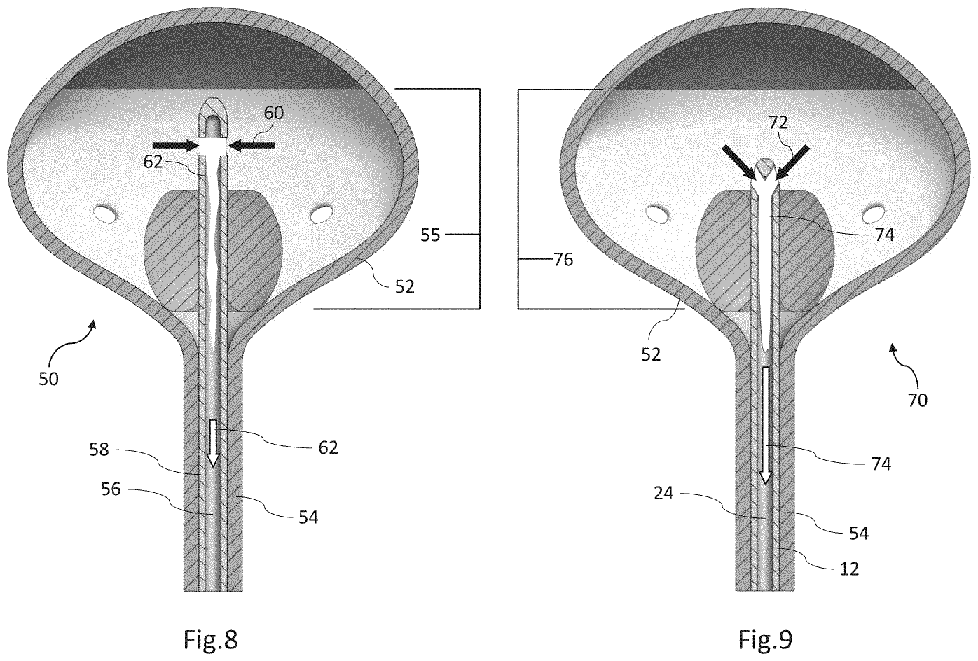

FIGS. 8-13 depict comparative differences between standard urinary catheters and the inventive indwelling urinary catheters 10, 30 in urine inflow vector into the inflow openings 20, the relative velocity of urine flow into and through the flow lumen, the resulting levels of urine remaining in the urinary bladder, and urine flow as the respective catheters are removed through the urethra.

FIG. 8 depicts a urine loaded bladder 50 with a standard indwelling urinary catheter 58, such as a Foley catheter, in its deployed state 50 in a urinary bladder 52 and passing through the bladder neck 54. The urine load 55 with the standard indwelling urinary catheter 58 is depicted in FIG. 8. It will be noted that the positioning of the inflow openings on lateral wall surfaces of catheter 58 are positioned toward an upper portion of the urine load 55 and positioned to create a urine inflow vector 60 that is substantially perpendicular to the longitudinal axis of catheter 58. This results in a mostly moderate flow of urine 62 into the inflow openings and through drainage lumen 56 of catheter 58.

In contradistinction to the standard indwelling urinary catheter 58, as illustrated in FIG. 9, illustrating a urine loaded bladder 70 with the inventive indwelling urinary catheter 12, when placed in bladder 52 through the bladder neck 54 has an inflow vector 72 through the inflow openings 20 that is angularly displaced between perpendicular to and parallel with the longitudinal axis of indwelling urinary catheter 12. The urine inflow openings 20 are positioned far closer to the distal end of the balloon than in the standard indwelling urinary catheter 58. The angular displacement of urine inflow vector 72 is between about 15 to about 75° and has a distal bias relative to the longitudinal axis of the indwelling urinary catheter. In this configuration, the urine flow from urine load 76 into and through the distal openings 20 forms a substantially continuous flow of urine into and through the drainage lumen 24 of indwelling urinary catheter 12. It will be appreciated that the velocity of urine flow 74 in a continuous robust flow circuit, such as that provided by the present invention, is greater than the moderate velocity of urine flow, as is found in standard indwelling urinary catheters.

In both the standard urinary catheters and the inventive urinary catheters, once the urine level in the bladder reaches the proximal end of the urine inflow openings 20, the urine flow will drain in a drop-wise fashion. If this condition remains for a period of time, solidification or crystallization of urine in the drainage lumen may occur. For example, during patient sleep cycles, the bladder will accumulate urine and with the patient in a horizontal position, the urine load level may not reach the level of the urine inflow openings 20 such that drainage will resume. As the patient assumes an upright position, the urine load will then reach the level of the urine inflow openings 20 and begin draining into the drainage lumen 24 A higher urine load level inside the bladder will result in a greater urine drainage flow velocity in the present invention, which will aid in flushing any solids or crystalline substances that may accumulate in the drainage lumen 24.

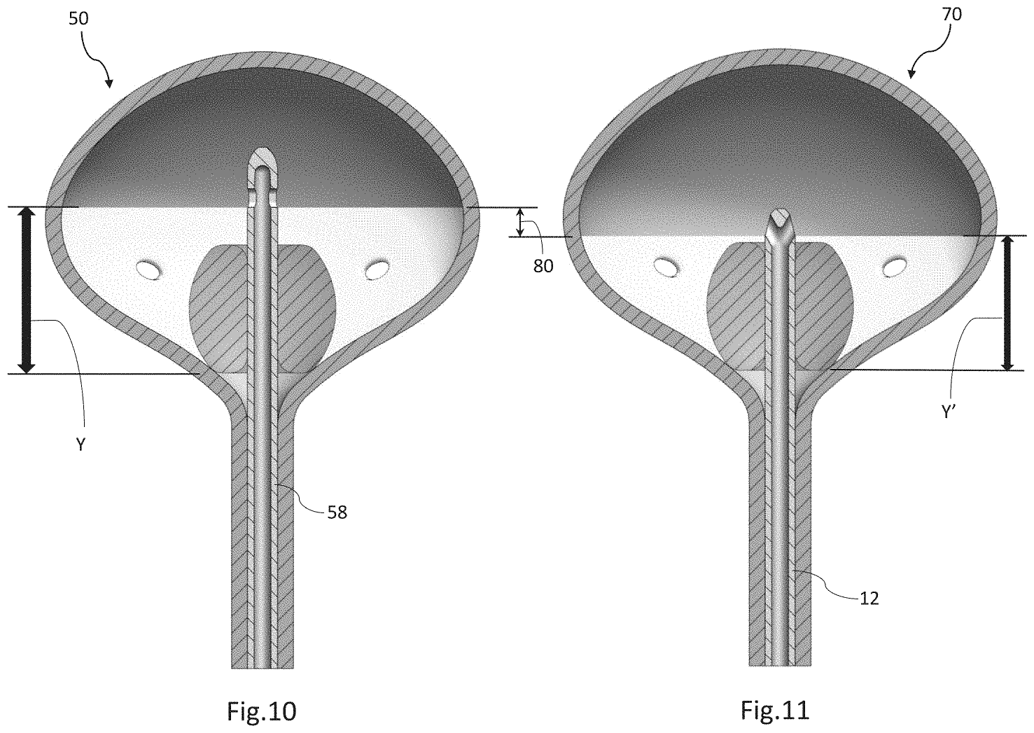

FIGS. 10, 11 and 14 depict a comparison of urine drainage between the standard urinary catheter 58 in a bladder 50 and the inventive urinary catheter 12 in a bladder 70, respectively, as measured by the level of urine, Y and Y′, remaining in the urinary bladder upon maximal emptying for each catheter. As will be seen in FIG. 10, because of the relatively greater distance between the proximal end of the balloon and the proximal end of inflow opening 20, urine in the bladder will only drain to a distance Y, leaving a relatively large volume of urine load in the bladder. In contrast, as illustrated in FIG. 11, the relatively closer proximity between the proximal end of balloon 14 and the proximal end of inflow openings 20, allows for a greater volume of urine drainage from the bladder and a corresponding smaller volume of urine load, as represented by Y′, remaining in the bladder. The difference between the remaining urine load with the standard urinary catheter 58 and the remaining urine load with the inventive urinary catheter 12 is represented by reference numeral 80 denoted between FIGS. 10 and 11.

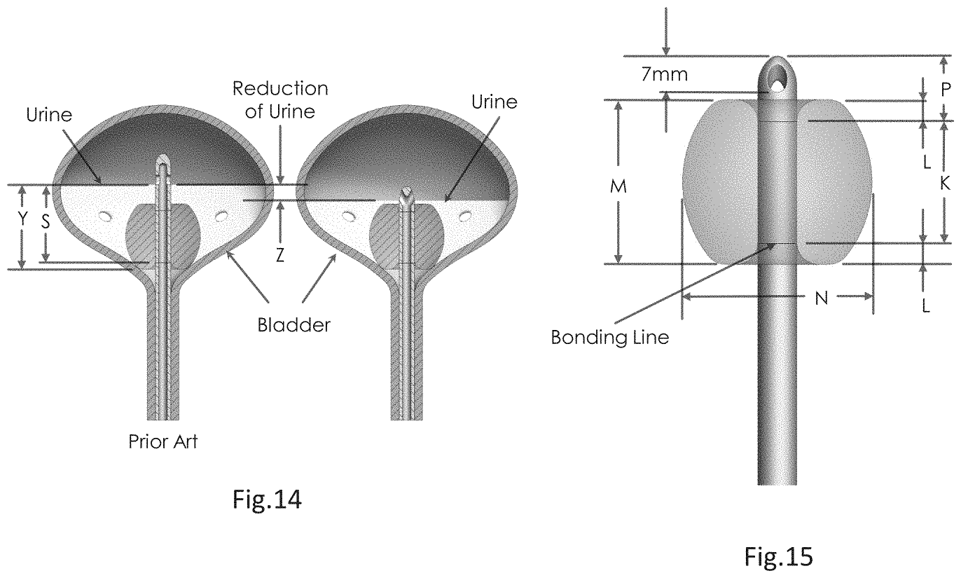

The same comparison is illustrated in FIG. 14, illustrating the difference Z between the remaining urine load in a standard urinary catheter 58 and that in the inventive urinary catheter 12 when drained to the level of the proximal end of urine inflow opening 20, respectively. The difference Z in remaining urine load is also influenced by balloon inflation volume as is illustrated in Table 1, below.

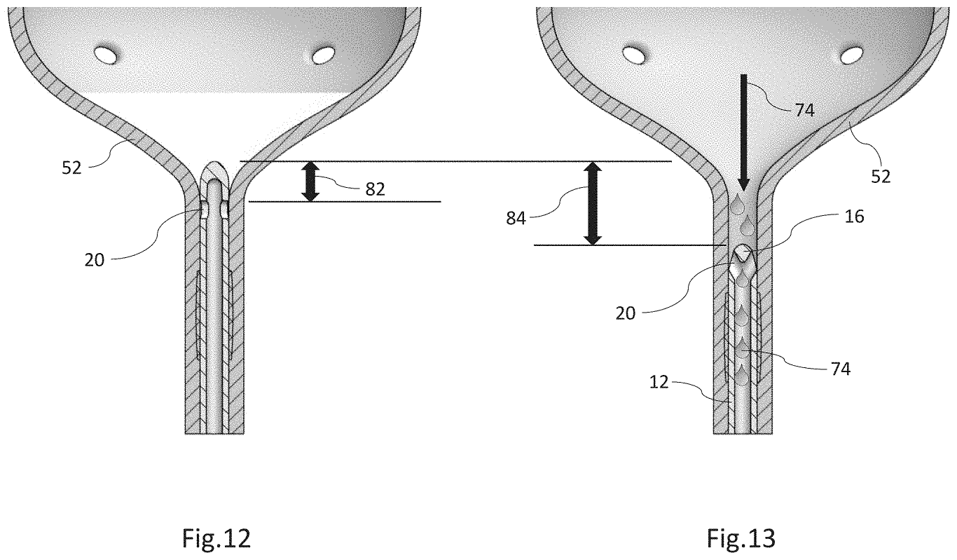

FIGS. 12 and 13 also illustrate a comparison between a standard indwelling urinary catheter 58 and the inventive indwelling urinary catheter 10, 30, respectively, during catheter withdrawal. In the standard indwelling urinary catheter 58, urine inflow ceases as soon the inflow openings 20 pass into the bladder neck a short distance 82 and are occluded by either the bladder neck tissue or the urethral tissue. In stark contrast, the distally biased orientation of the inflow openings 20 in the inventive indwelling urinary catheter 12, causes the inflow openings 20 to remain open and not become occluded by either the bladder neck or urethra as the catheter is being withdrawn even a relatively large distance 84, as compared to the standard indwelling urinary catheter 58. In this manner, urine flow 74 from the bladder and into the inventive indwelling urinary catheter 10, 30, remains continuous until either the muscles of the bladder or the bladder neck contract to seal off the bladder from the urethra.

Thus, with respect to the Foley catheter, the present invention is advantageous in reducing the total length of the catheter inside the bladder. In one example of the embodiments of the invention, using the equation ((T+L)−(M+7)), it can be calculated the catheter is capable of reducing the total catheter length inside the bladder by 17 mm for the two-way embodiment and 19 mm for the three-way embodiment when compared with a standard Foley catheter. Values of L and M are shown in Table 1, below. The risk of damage to the bladder wall due to the distal tip impacting the bladder during patient movement or insertion is minimized as a result of the catheter tip being far shorter than that of a Foley catheter. The present invention increases the volume of urine drainage and, therefore, reduces the level of urine remaining in the bladder by at least 8 mm for the two-way catheter embodiment 10 and about 10 mm for the three-way embodiment 30, both relative to the Foley catheter, as shown in Table 1.

Due to the generally ovular shape of the bladder, its widest dimension is intermediate the superior or inferior aspects of the bladder. Therefore, a reduction in the height of the urine load within the bladder does not directly correlate with the volume of urine drained from the bladder. A urine load height reduction of 1 mm at the center of the bladder is a greater reduction than a similar 1 mm urine load height reduction at the superior or inferior aspects of the bladder, both of which have smaller transverse cross-sectional areas. Based on that observation, a reduction of value of 8 mm or 10 mm in urine load height may present an empty bladder, except for residual urine around the balloon 14. Upon substantial drainage of urine from the bladder, any residual urine will have a very small volume due to its being located in the inferior aspect of the bladder. The height of a residual urine load in the bladder will depend upon the balloon capacity, which should not exceed 33 mm.

Under normal conditions, the bladder's muscular contractions will occur when the micturition response occurs, typically when the bladder is full and there is a need for urination. In this case, the muscular contractions of the bladder will force urine down into the urethral opening for drainage and an empty bladder. Where an indwelling urinary catheter is placed in the bladder, the patients discomfort occurs mostly from two factors: balloon size and a greater than normal amount of urine remaining in the bladder. These factors typically cause the urge to urinate more frequently. The muscular contraction of the bladder will raise the urine level around the balloon 14 periodically as will motion, such as walking or laying down. As these events occur, the urine will drain into the urine inflow openings 20 and drain into the urinary catheter 10. Because the bottom of the urine inflow openings 20 are positioned at substantially the same level as or below the superior balloon surface, urine will flow more readily and faster into the urine inflow openings 20. This will lead to at least substantially complete urine drainage from the bladder.

Moreover, the configuration of the inflow openings 20 both increases the urine flow rate, reduces the risk of solidification and blocking the catheter, eliminates the risk of the bladder wall being sucked into the inflow openings, and allows for drainage of urine as a result of the patient's normal movements. Further, as the balloon is collapsed and the catheter is being removed from the bladder, the flow of urine into the inflow openings 20 will continue until as long as the urethra remains patent during withdrawal. Where the balloon is partially collapsed, the muscular contractions of the bladder may drive the catheter into the urethra and, in conventional urinary catheters, either the balloon or the urethral walls will occlude the urine inflow openings 20 and cause stoppage of the urine into the drainage lumen. In the present invention, urine flow is maintained despite either full or partial collapse of the balloon or mispositioning of the catheter in the urethra. Overall, the foregoing advantages of the present invention will result in easier insertion into and through the urethra and into the bladder and less patient irritation and discomfort due to the greater bladder drainage and the increased velocity of urine flow into the drainage lumen.

When compared to the FLUME catheter, the present invention advantageously maintains urine flow when the balloon is collapsed, and the catheter is being withdrawn from the bladder and urethra. Further, because the balloon of the FLUME catheter is of a relatively large size and has two recesses or channels in the exterior surface of the balloon to channel urine to the urine inflow opening, that require the balloon to be folded over the distal tip, the risk of urine flow blockage is far greater than that with the present invention, leading to a greater risk of patient discomfort and irritation.

### Example

A study was performed to determine the impact of balloon capacity and balloon shape when inflated on the life span of the indwelling inflated balloon, the amount of urine remaining inside the bladder after drainage, balloon leakage and collapse, and patient acceptance. Five (5) 24 Fr Foley Catheters having a silicon-elastomer coated latex balloon with an inflation capacity of 30 ml, of both two-way and three-way designs, were measured for physical dimensions of the following:

Distance between two bonding lines of the balloon to the catheter; Measurement “K”;

Height of the inflated balloon; Measurement “M=K+2L”;

Extended length of the balloon beyond each bonding line; Measurement “L”;

Outer diameter of the inflated balloon; Measurement “N”;

Distance between the distal tip of the inventive catheter to the distal bonding line; Measurement “P=L+7 mm”; The dimensions in column “P” are intended to be exemplary and presented only as basic guidance to one skilled in the art to practice the present invention.

Distance between the bottom of the urine inflow opening to the proximal bonding balloon prior to balloon inflation; Measurement “S”;

Distance from the bottom of the each urine inflow opening to the proximal end of the inflated balloon at 30 ml inflation capacity in a standard Foley catheter; Measurement “Y=S+L”;

Distance from the proximal end of urine inflow opening 20 to the proximal end of the inflated balloon at 30 ml inflation capacity in the inventive catheter; Measurement “Y=(S+L)=(K+2L)=M”;

Distance from proximal end of urine inflow opening to the distal end of the distal tip in the inventive catheter; Measurement “X”;

Height of urine reduction inside the bladder; Measurement “Z=Y−M”; and

Ratio of urine reduction inside the bladder; Measurement “%=Z/Y”.

FIGS. 14 and 15 graphically illustrate each of the foregoing measurements relative to the standard Foley catheter and the inventive indwelling urinary catheter depicted in FIGS. 14 and 15. In the inventive indwelling catheter in FIG. 15, distance λ was a constant 7 mm for purposes of calculated comparisons with the standard Foley catheter depicted in FIG. 14. The 7 mm distance X was selected to maintain a constant distance for the urine drain openings 20 in order to reduce interference between the distal tip of the catheter and the urethra and facilitate more comfortable insertion of the catheter through the urethra and into the bladder. Table 1, below, presents the calculated data for the above measurements as a function of different water inflation volume in the 30 ml capacity balloons in 24 Fr Foley urinary catheters.

TABLE 1

| Balloon | | | | | | S | | Y | | Z | | | |
|---|---|---|---|---|---|---|---|---|---|---|---|---|---|
| Inflated | | | | | | (mm) | | (mm) | | (mm) | | % | |
| Volume | K | M | L | N | P | 2- | 3- | 2- | 3- | 2- | 3- | 2- | 3- |
| (ml) | (mm) | (mm) | (mm) | (mm) | (mm) | WAY | WAY | WAY | WAY | WAY | WAY | WAY | WAY |
| 30 | 25 | 33 | 4.0 | 39 | 11.0 | 37 | 39 | 41.0 | 43.0 | 8.0 | 10.0 | 20% | 23% |
| 25 | 25 | 32 | 3.5 | 36 | 10.5 | 37 | 39 | 40.5 | 42.5 | 8.5 | 10.5 | 21% | 25% |
| 20 | 25 | 30 | 2.5 | 34 | 9.5 | 37 | 39 | 39.5 | 41.5 | 9.5 | 11.5 | 24% | 28% |
| 15 | 25 | 26 | 0.5 | 30 | 7.5 | 37 | 39 | 37.5 | 39.5 | 11.5 | 1.5 | 31% | 34% |
| 10 | 25 | 23 | 0.0 | 26 | 7.0 | 37 | 39 | 37.0 | 39.0 | 14.0 | 16.0 | 38% | 41% |

At 10 ml inflation volume, the balloon was not inflated to its full geometry. In this cause, the height of the inflated balloon (M) was 23 mm while the height between the two bonding lines (K) was 25 mm. This resulted in the inflated balloon being 2 mm shorter than the bonding line length. In that case, the final step for securing the catheter in the bladder would be to pull out the catheter 2 mm to seat the balloon in the bladder neck, which will slightly increase the ability of the catheter to reduce the volume of the urine load within the bladder. Some clinicians prefer to use a 10 ml inflation capacity primarily to increase patent comfort.

The height of urine reduction inside the bladder (Z=Y−M) was calculated based upon the difference in distance between the bottom of the urine inflow opening in the Foley Catheter and the same measurement for the inventive catheter described herein.

The Foley catheter balloon may be modified to change the distance between two bonding lines of the balloon to the catheter (K) from 25 mm to 20 mm with the potential result in an additional approximately 3 mm of reduction in the urine level within the bladder after drainage (Z). A change in balloon length to 20 mm, together with the use of smaller inflation balloon capacities, may achieve some benefits similar to that of using a smaller length or diameter balloon. This change, however, requires changes in manufacturing processes.

The data suggest that an inflated balloon with a capacity of 15 ml, rather than the 30 ml capacity of the Foley catheters, most improves urine drainage. This same capacity reduces the hydrostatic stress on the control valve by about 50% relative to a 30 ml inflation volume, and the balloon at lower inflation volumes has less hydrostatic pressure on the balloon material, allowing it to be more pliable when in the bladder and able to accept normal micturition contractions of the bladder without adverse effects on the inflation control valve and/or the balloon wall itself. These factors result in increased lifespan and durability of the catheter balloon in the bladder, increased patient comfort and reduced patient irritation lending itself to a higher degree of patient compliance with the indwelling catheter and, therefore, allow the patient to have a more rapid recovery.

While the present invention has been described with reference to specific exemplary embodiments, it will be evident that various modifications and changes may be made to these embodiments without departing from the broader spirit and scope of the invention as set forth in the claims. Accordingly, the specification and drawings are to be regarded in an illustrative rather than a restrictive sense.

1. An indwelling urinary catheter having a catheter body, a balloon, a balloon inflation lumen, a common drainage lumen, and at least two diametrically opposed urine inflow openings at a distal end of the catheter body each of the urine inflow openings in fluid flow communication with the common drainage lumen, wherein the urine inflow openings have a distally facing bias with an opening orientation that is angularly displaced with an angle α between about 15 degrees to about 75 degrees relative to a longitudinal axis of the catheter body, wherein the catheter body further includes a substantially frustoconical distal tip and the at least two diametrically opposed urine inflow openings pass into the substantially frustoconical distal tip forming a substantially Y-shaped opening in fluid flow communication with the drainage lumen and are configured to convey urine flow into both of the at least two diametrically opposed urine inflow openings simultaneously and remain open to urine inflow when the balloon is in both its inflated and deflated states and when the distal tip is positioned in the urinary bladder or urethra.

2. (canceled)

3. The indwelling urinary catheter of claim 1, wherein the urine inflow openings are in proximity to a distal end of the balloon when the balloon is in both its inflated and its deflated states.

4. The indwelling urinary catheter of claim 3, wherein at least one of the urine inflow openings has a proximal end that is substantially even with or below the distal end of the balloon.

5. (canceled)

6. The indwelling urinary catheter of claim 1, wherein angle α has a value between about 20 to about 45°.

7. The indwelling urinary catheter of claim 1, wherein angle α has a value between about 25 to about 35°.

8. (canceled)

9. (canceled)

10. The indwelling urinary catheter of claim 1, wherein a proximal end of each of the at least two inflow openings are about 7 mm from a distal end of frustoconical tip.

11. The indwelling urinary catheter of claim 10, wherein a distance from a proximal end of the balloon to a proximal end of each of the at least two urine inflow openings is between about 25 to about 29 mm.

12. The indwelling urinary catheter of claim 11, wherein a distance between a proximal end of the balloon and a distal end of the catheter body is between about 32 to about 36 mm.

13. The indwelling urinary catheter of claim 1, further comprising a third lumen in fluid flow communication with a port passing through a distal end of the catheter body.

14. The indwelling urinary catheter of claim 13, wherein the port passes through a distal tip of the catheter body.

15. The indwelling urinary catheter of claim 14, wherein the distal tip further comprises a generally frustoconical shape and the port passes through a lateral wall surface of the distal tip and is spaced apart from the urine inflow openings.

16. An indwelling urinary catheter, comprising a catheter body having a substantially conical distal tip, an inflatable balloon in proximity to the substantially conical distal tip, a balloon inflation lumen, a balloon inflation connector, a drainage lumen, a drainage port, and at least two urine inflow openings passing through lateral wall surfaces of the distal tip, wherein each of the at least two urine inflow openings have an angle between about 20 to about 45° relative to a longitudinal axis of the catheter body.

17. The indwelling urinary catheter of claim 16, wherein each of the at least two urine inflow openings have a substantially circular-shaped opening profile.

18. The indwelling urinary catheter of claim 17, wherein a proximal end of each of the at least two urine inflow openings are substantially even with or below a distal end of the inflatable balloon.

19. The indwelling urinary catheter of claim 16, further comprising a third lumen in the catheter body which is in fluid flow communication with a port passing through the substantially conical distal tip.

20. The indwelling urinary catheter of claim 19, wherein the port is positioned in spaced apart relationship from the at least two urine inflow openings.

21. An indwelling urinary catheter having a catheter body, a balloon, a balloon inflation lumen, a drainage lumen, and opposing urine inflow openings at a distal end of the catheter body in fluid flow communication with the drainage lumen, comprising, at least one fluid conduit passing between the opposing urine inflow openings and the drainage lumen, the fluid conduit having an angle α between about 15 degrees to about 75 degrees relative to a longitudinal axis of the catheter body such that the at least one fluid conduit and the drainage lumen for a substantially Y-shaped fluid conduit, wherein the catheter body further includes a substantially frustoconical distal tip and the opposing urine inflow openings pass laterally through the wall surfaces and are in fluid flow communication with the fluid conduit and the drainage lumen and are configured to convey urine flow into both of the opposing urine inflow openings simultaneously and the opposing urine inflow openings remain open to urine inflow when the balloon is in both its inflated and deflated states and when the frustoconical distal tip is in the urinary bladder or urethra.

22. The indwelling urinary catheter of claim 21, further comprising a teritiary lumen configured to deliver or withdraw fluids from an opening at the frustoconical distal tip.

23. The indwelling urinary catheter of claim 1, wherein the frustoconical distal tip is configured to remain substantially co-axial with a longitudinal axis of the catheter body when the balloon is in an inflated state.

24. The indwelling urinary catheter of claim 1, wherein a distance between a distal end of the balloon in an inflated state and a proximal end of the urinary inflow openings is between about 0 mm to about 4 mm.

25. The indwelling urinary catheter of claim 21, wherein the frustoconical distal tip is configured to remain substantially co-axial with a longitudinal axis of the catheter body when the balloon is in an inflated state.

26. The indwelling urinary catheter of claim 21, wherein a distance between a distal end of the balloon in an inflated state and a proximal end of the urinary inflow openings is between about 0 mm to about 4 mm.
